(19) United States
(12) Patent Application Publication
(10) Pub. No.: US 2011/0250192 A1
(43) Pub. Date: Oct. 13, 2011

(54) USE OF MICRORNA SIGNATURES FOR ASSESSING RISK LEVELS OF NEUROBLASTOMA PATIENTS

(75) Inventors: Alice L. Yu, Taipei City (TW); You-Chin Lin, Taipei City (TW); Ruey-Jen Lin, Yilan City (TW)

(73) Assignee: Academia Sinica, Taipei (TW)

(21) Appl. No.: 13/056,043

(22) PCT Filed: Aug. 3, 2009
(86) PCT No.: PCT/US2009/052550

§ 371 Date: Jun. 14, 2011

Related U.S. Application Data
(60) Provisional application No. 61/137,653, filed on Aug. 1, 2008.

(51) Int. Cl. A61K 38/46; A61K 48/00; A61P 35/00; C12Q 1/68

(52) U.S. Cl. 424/94.6; 435/6.1; 435/6.12; 514/44 R

(57) ABSTRACT

Methods for assessing the risk level or survival/death probability of a neuroblastoma patient based on a number of microRNA signatures, optionally in combination with Dicer, Drosha, and age at diagnosis. Also disclosed herein is use of Dicer, Drosha, or both in suppressing neuroblastoma cell growth

### CROSS-REFERENCE TO RELATED APPLICATION PARAGRAPH

This application claims the benefit of U.S. Provisional Application No. 61/137,653, filed on Aug. 1, 2008, the contents of which is hereby incorporated by reference in its entirety

### BACKGROUND OF THE INVENTION

Neuroblastoma, accounting for 15% of pediatric cancer deaths, is a common childhood tumor derived from primitive sympathetic neuroblasts. Based on its plethoric clinical behavior, neuroblastoma can be categorized into two risk groups. High-risk neuroblastoma undergoes malignant tumor progression, while low-risk neuroblastoma either regresses spontaneously or differentiates into benign ganglioneuroma. To achieve high therapeutic efficacy, different treatments shall be applied to patients bearing neuroblastoma tumors with different risk levels.

MicroRNAs (miRNAs) are a class of small noncoding RNAs that negatively regulate gene expression. These small RNAs are initially produced in cells as long precursors, which are then processed to generate mature miRNAs. Dicer and Drosha are two major endonucleases involved in miRNA processing. miRNAs have been found to play important roles in many physiological processes related to cancer development, e.g., cell proliferation, apoptosis, and differentiation. It has been suggested that miRNAs may serve as prognostic markers and therapeutic targets in cancer treatment.

### SUMMARY OF THE INVENTION

The present invention is based, at least in part, on unexpected discoveries that certain miRNA signatures, optionally in combination with other factors (i.e., Dicer, Drosha, and age at diagnosis), are closely associated with a neuroblastoma patient's risk level or survival/death probability.

In one aspect, the present invention features a method of determining the risk level of a neuroblastoma patient based on a 15-biomarker signature, including 12 microRNAs, Dicer, Drosha, and age at diagnosis. This method includes the following steps: (i) obtaining a set of data indicating the expression levels of 12 microRNAs hsa-miRNAs-29a, hsa-miRNAs-30c, hsa-miRNAs-30e, hsa-miRNAs-95, hsa-miRNAs-128a, hsa-miRNAs-128b, hsa-miRNAs-135a, hsa-miRNAs-135b, hsa-miRNAs-137, hsa-miRNAs-138, hsa-miRNAs-148a, and hsa-miRNAs-195 in a neuroblastoma sample of the patient, the expression levels of Dicer and Drosha in the sample, and the patient's age at diagnosis, (ii) processing the set of data by computational analysis to determine a risk pattern, and (iii) assessing the patient's risk level based on the risk pattern. When a patient is determined to exhibit risk pattern A, C, and D, it indicates that the patient has a high risk level, a low risk level, and a medium-to-low risk level, respectively. In this method, the expression levels of the 12 miRNAs, Dicer, and Drosha can be determined by real-time PCR.

In one example, this method is applied to a patient who has not been subjected to clinical staging or any other risk assessment.

In another aspect, this invention features a method of assessing the risk level of a neuroblastoma patient based on a 27-miRNA signature. This method includes (i) obtaining a set of data indicating the expression levels of 27 microRNAs, including hsa-miR-149, hsa-miR-129, hsa-miR-27b, hsa-miR-23b, hsa-miR-190, hsa-miR-128a, hsa-miR-15a, hsa-miR-148a, hsa-miR-137, hsa-miR-30c, hsa-miR-197, hsa-miR-195, hsa-miR-26b, hsa-miR-21, hsa-miR-30b, hsa-miR-135a, hsa-miR-126, hsa-miR-95, hsa-miR-142-5p, hsa-miR-128b, hsa-miR-98, hsa-miR-142-3p, hsa-miR-340, hsa-miR-30e, hsa-miR-331, hsa-miR-140, and hsa-miR-324-5p, in a neuroblastoma sample of a patient, (ii) processing the set of data by computational analysis to determine a microRNA signature that characterizes the expression profile of the 27 microRNAs, and (iii) assessing the risk level of the patient based on the 27-miRNA signature. A signature representing low expression of the 27 miRNAs indicates that the patient is a high-risk neuroblastoma patient and a signature representing high expression of the miRNAs indicates that the patient is a low-risk neuroblastoma patient. In one example, the computational analysis is Prediction Analysis of Microarray (PAM) analysis.

In yet another aspect, this invention provides a method of assessing the risk level of a neuroblastoma patient based on a miRNA signature of a neuroblastoma patient. This miRNA signature is determined based on the expression level(s) of one or more of the following miRNAs: hsa-miR-23b, hsa-miR-128a, hsa-miR-15a, hsa-miR-148a, hsa-miR-197, hsa-miR-195, hsa-miR-26b, hsa-miR-21, hsa-miR-135a, hsa-miR-126, hsa-miR-95, hsa-miR-142-5p, hsa-miR-128b, hsa-miR-98, hsa-miR-142-3p, hsa-miR-340, hsa-miR-140. Alternatively, the assessment is made based on a miRNA signature including (a) one or more miRNAs listed above, and (b) one or more of the miRNAs listed below: hsa-miR-149, hsa-miR-129, hsa-miR-27b, hsa-miR-190, hsa-miR-137, hsa-miR-30c, hsa-miR-30b, hsa-miR-30e, hsa-miR-331, and hsa-miR-324-5p. When a miRNA signature of a neuroblastoma patient, determined by computational analysis, represents low expression of the constituting miRNAs, the patient is assessed as a high-risk patient. On the other hand, when the miRNA signature represents high expression of the constituting miRNAs, it indicates that the patient is a low-risk neuroblastoma patient.

The present invention also provides a method for predicting a neuroblastoma patient's survival/death probability based on a 20-miRNA signature including hsa-miR-26a, hsa-miR-26b, hsa-miR-27b, hsa-miR-30a-3p, hsa-miR-30e, hsa-miR-95, hsa-miR-128a, hsa-miR-128b, hsa-miR-129, hsa-miR-137, hsa-miR-146, hsa-miR-148a, hsa-miR-149, hsa-miR-152, hsa-miR-186, hsa-miR-190, hsa-miR-197, hsa-miR-324-5p, hsa-miR-331, and hsa-miR-335. If a patient displays a signature characterizing low expression of these miRNAs, that patient is predicted to have a high survival probability. On the other hand, if a patient has a signature characterizing high expression of these miRNAs, he or she is predicted to have a low survival rate.

In still another aspect, the invention features a risk assessment method based on the expression level profile of Dicer, Drosha, or both, as determined by computational analysis, in a neuroblastoma sample of a patient. A profile representing low expression of Dicer, Drosha, or both indicates that the patient has a high risk level and a profile representing low expression of these two proteins indicates that the patient has a low risk level. In one example, the expression level profile of Dicer is determined to assess the risk level of a patient bearing a neuroblastoma tumor with no MYCN amplification.

Also within the scope of this invention is a method of inhibiting neuroblastoma cell growth by administering to a neuroblastoma patient an effective amount of a composition containing (i) a polypeptide including the amino acid sequence of Dicer or Drosha, or a nucleotide sequence encoding the polypeptide, and (ii) a pharmaceutically acceptable carrier. “An effective amount” as used herein refers to the amount of each active agent required to confer therapeutic effect on the patient, either alone or in combination with one or more other active agents. Effective amounts vary, as recognized by those skilled in the art, depending on route of administration, excipient choice, and co-usage with other active agents. The just-described composition can also be used in manufacturing a medicament for inhibiting neuroblastoma cell growth.

The details of one or more embodiments of the invention are set forth in the description below. Other features or advantages of the present invention will be apparent from the following drawings and detailed description of several examples, and also from the appended claims.

### BRIEF DESCRIPTION OF THE DRAWINGS

The drawings are first described.

FIG. 1 shows four risk patterns, Pattern A, Pattern B, Pattern C, and Pattern D, which are determined by PNNSolution analysis based on 15 biomarkers, i.e., 12 miRNAs hsa-miRNAs-29a, hsa-miRNAs-30c, hsa-miRNAs-30e, hsa-miRNAs-95, hsa-miRNAs-128a, hsa-miRNAs-128b, hsa-miRNAs-135a, hsa-miRNAs-135b, hsa-miRNAs-137, hsa-miRNAs-138, hsa-miRNAs-148a, and hsa-miRNAs-195, Dicer, Drosha, and age at diagnosis. A: risk patterns A, B, C, and D. B: distribution of 66 neuroblastoma patients characteristic for each of the four risk patterns and Kaplan-Meier estimates of overall survival of the 66 patients according to their risk patterns.

FIG. 2 includes charts showing the correlations between Dicer/Drosha expression levels and survival/death probability of neuroblastoma patients. A: correlation between Dicer and probability of event-free survival or overall survival in neuroblastoma patients. B: correlation between Drosha and probability of event-free survival or overall survival in neuroblastoma patients. C: correlation between Dicer and probability of event-free survival or overall survival in neuroblastoma patients with no MYCN amplification. D: correlation between Drosha and probability of event-free survival or overall survival in neuroblastoma patients with no MYCN amplification.

FIG. 3 includes tables summarizing correlations between various clinical factors and event-free survival (table A) or overall survival (table B) in neuroblastoma patients via univariate and multivariate Cox regression analysis.

FIG. 4 includes tables summarizing correlations between various clinical factors and event-free survival (table A) or overall survival (table B) in neuroblastoma patients with no MYCN amplification via univariate and multivariate Cox regression analysis.

FIG. 5 is a diagram showing down-regulation of Dicer and Drosha via RNA interference. A: a photo showing reduced expression of Dicer and Drosha, as determined by Westernblot, in neuroblastoma cells Be2C, NMB7, and NB5 transfected with plasmids expressing shRNAs targeting Dicer and Drosha. B; a chart showing reduced expression of Dicer and Drosha, determined by quantitative RT-PCR, in the same transfected neuroblastoma cells. C: a chart showing down-regulation of miRNA hsa-let7a and hsa-mir-17-5p in the transfected neuroblastoma cells.

FIG. 6 is a diagram showing that down-regulation of Dicer and Drosha via RNA interference promotes neuroblastoma cell growth. (a): showing survival rates of neuroblastoma cells transfected with plasmids expressing shRNAs targeting Dicer and Drosha. (b): showing colony formation rates of the transfected neuroblastoma cells in soft agar.

FIG. 7 is a table summarizing survival/death probability prediction based on a 20-miRNA signature including hsa-miR-26a, hsa-miR-26b, hsa-miR-27b, hsa-miR-30a-3p, hsa-miR-30e, hsa-miR-95, hsa-miR-128a, hsa-miR-128b, hsa-miR-129, hsa-miR-137, hsa-miR-146, hsa-miR-148a, hsa-miR-149, hsa-miR-152, hsa-miR-186, hsa-miR-190, hsa-miR-197, hsa-miR-324-5p, hsa-miR-331, and hsa-miR-335.

### DETAILED DESCRIPTION OF THE INVENTION

Neuroblastoma patients can be categorized into two risk groups, i.e., high-risk and low-risk, based on the behaviors of the tumor they have. See Maris et al., Neuroblastoma Lancet 369(9579):2106-2120, 2007. A neuroblastoma patient's risk level is closely associated with clinical stages and survival/death rates. Tables 1 and 2 below show the different neuroblastoma stages (under the International Neuroblastoma Staging System and the International Neuroblastoma Risk Group Staging System) and their correlations with risk levels:

TABLE 1
Neuroblastoma Clinical Stages

International Neuroblastoma Staging System (INSS):
Stage 1: Localized tumor confined to the area of origin.
Stage 2A: Unilateral tumor with incomplete gross resection; identifiable
ipsilateral and contralateral lymph node negative for tumor.
Stage 2B: Unilateral tumor with complete or incomplete gross resection;
with ipsilateral lymph node positive for tumor; identifiable contralateral
lymph node negative for tumor.
Stage 3: Tumor infiltrating across midline with or without regional lymph
node involvement; or unilateral tumor with contralateral lymph node
involvement; or midline tumor with bilateral lymph node involvement.
Stage 4: Dissemination of tumor to distant lymph nodes, bone marrow,
bone, liver, or other organs except as defined by Stage 4S.
Stage 4S: Age <1 year old with localized primary tumor as defined in
Stage 1 or 2, with dissemination limited to liver, skin, or bone marrow
(less than 10 percent of nucleated bone marrow cells are tumors).
International Neuroblastoma Risk Group Staging System (INRGSS):
Stage L1: Localized disease without image-defined risk factors.
Stage L2: Localized, disease with image-defined risk factors.
Stage M: Metastatic disease.
Stage MS: Metastatic disease “special” where MS is equivalent to
stage 4S.

TABLE 2
Neuroblastoma Stages and Risk Levels

| | Age | | MYCN | Ploidy | Histology | Other | Risk group |
|---|---|---|---|---|---|---|---|
| 1 | | | | | | | Low |
| 2A/2B | | | Not amplified | | | >50% resection | Low |
| | | | Not amplified | | | <50% resection | Intermediate |
| | | | Not amplified | | | Biopsy only | Intermediate |
| | | | Amplified | | | | High |
| 3 | <547 | days | Not amplified | | | | Intermediate |
| | ≧547 | days | Not amplified | | Favourable | | Intermediate |
| | | | Amplified | | | | High |
| | ≧547 | days | Not amplified | | Unfavourable | | High |
| 4 | <365 | days | Amplified | | | | High |
| | <365 | days | Not amplified | | | | Intermediate |
| | 365-547 | days | Amplified | | | | High |
| | 365-547 | days | | DI = 1 | | | High |
| | 365-547 | days | | | Unfavourable | | High |
| | 365-547 | days | Not amplified | DI > 1 | Favourable | | Intermediate |
| | ≧547 | days | | | | | High |
| 4S | <365 | days | Not amplified | DI > 1 | Favourable | Asymptomatic | Low |
| | <365 | days | Not amplified | DI = 1 | | | Intermediate |
| | <365 | days | Missing | Missing | Missing | | Intermediate |
| | <365 | days | Not amplified | | | Symptomatic | Intermediate |
| | <365 | days | Not amplified | | Unfavourable | | Intermediate |
| | <365 | days | Amplified | | | | High |

Data do not change risk grouping.
Table: Proposed Children’s Oncology Group risk stratification schema, by stage

To achieve the best therapeutic efficacy, different approaches should be employed for treating neuroblastoma patients in different clinical stages or having different risk levels. Thus, it is of particular importance to assess a neuroblastoma patient's risk level so as to determine the optimal treatment for that patient.

### Assessing Risk Levels Based on MiRNA Signatures

We have discovered that certain miRNA signatures, characterizing the expression level profiles of one or more miRNAs, are reliable markers for assessing a neuroblastoma patient's risk level. More specifically, (a) the 27 miRNAs described in Example 1 below are differentially expressed in high-risk neuroblastoma patients versus low-risk patients and therefore any of the 27 miRNAs or a combination thereof serves as a marker for determining a patient's risk level, (b) the 27 miRNAs as a whole constitute a reliable miRNA signature for assessing the risk level of a neuroblastoma patient (see Example 2 below), and (c) a miRNA signature including the 20 miRNAs described in Example 6 below serves as a reliable marker for predicting a neuroblastoma patient's survival/death probability.

Accordingly, the present invention relates to a method to assess a neuroblastoma patient's risk level or survival/death probability based on any of the miRNA signatures mentioned above.

To practice this method, a neuroblastoma tumor sample is obtained from a patient (e.g., a Caucasian, an Asian, an African, or a Hispanic) and the expression level(s) of the miRNA(s) that constitutes a miRNA signature of interest can be determined by conventional methods. In one example, the expression levels are determined by quantitative PCR (also known as real-time PCR) using a kit containing a set of primers specific to the miRNAs to be analyzed. The kit can further contain a pair of primers specific to an internal control RNA, e.g., U6 snRNA. The data indicating miRNA expression levels is first normalized against the expression level of the control RNA and the normalized data is then processed by a computational program to generate a miRNA signature (e.g., represented by a numeric number) that characterizes the expression level profile of the miRNAs. This signature is compared with a reference point to determine whether it represents low expression or high expression of the miRNAs. The reference point can be determined based on the miRNA signatures including the same miRNAs obtained from high-risk and low-risk neuroblastoma patients via computational analysis. For example, it can be the middle point between the signature of high-risk patients and the signature of low-risk patients. When the signature represents low expression of the miRNAs (i.e., similar to that obtained from high-risk neuroblastoma patients), it indicates that the patient has a high risk level. One the other hand, when the signature represents high expression of the miRNAs (i.e., similar to that obtained from low-risk neuroblastoma patients), that patient is determined to have a low risk level.

Various computational programs can be applied in the method of this invention. Examples include, but are not limited to, Prediction Analysis of Microarray (PAM; see Tibshirani et al., PNAS 99(10):6567-6572, 2002); Plausible Neural Network (PNN; see, e.g., U.S. Pat. No. 7,287,014), PNNSulotion software and others provided by PNN Technologies Inc., Woodbridge, Va., USA, and Significance Analysis of Microarray (SAM).

### Assessing Risk Levels Based on 15 Biomarkers

We have further discovered that a 15-Biomarker signature, including the 12 miRNAs described in Example 4 below, Dicer, Drosha, and age at diagnosis, is a reliable marker for assessing the risk level of a neuroblastoma patient, in particular, a patient free of clinical staging or any other risk assessment (e.g., MYCN amplification or Shimada histology).

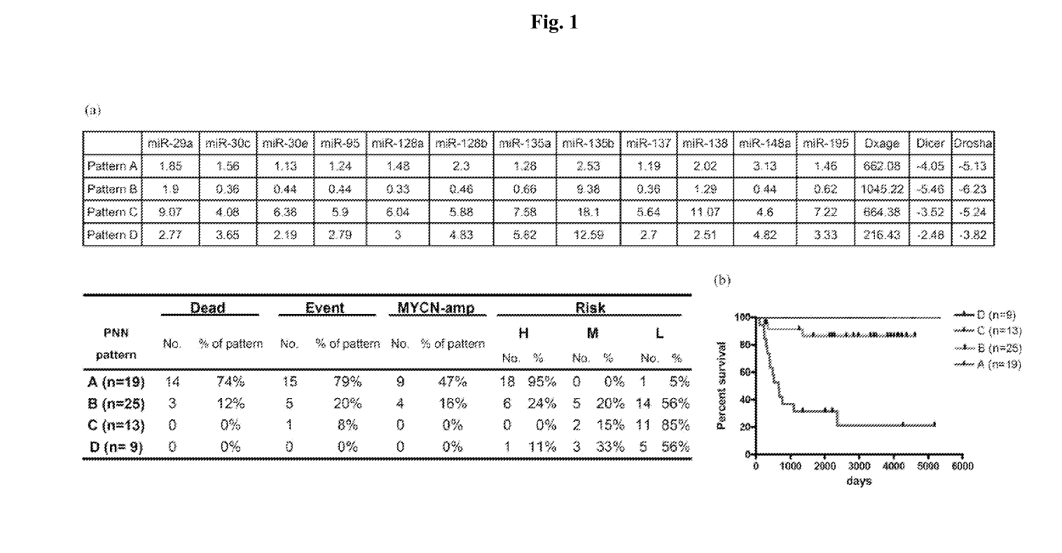

Accordingly, the present invention features a risk assessment method using the 15-Biomarker signature as an indicator. The expression levels of the 12 miRNAs, Dicer, and Drosha in the neuroblastoma of a patient can be determined based on the method described above. The data indicating their expression levels and the patient's age at diagnosis are processed by a computational program, e.g., PNNSolution, to produce a risk pattern for that patient. This risk pattern can be compared with pre-determined risk patterns representing particular risk levels to determine the patient's risk level. For example, if the risk pattern falls in Pattern A, Pattern C, and Pattern D shown in FIG. 1, it indicates that the patient is at high risk, low risk, and medium-to-low risk, respectively. A patient displaying Pattern C or D has a high survival rate. If the risk pattern falls in Pattern B also shown in FIG. 1, it indicates that the patient is likely to be at low-to-medium risk. Further risk assessment would be need to accurately determine that patient's risk level.

### Assessing Risk Levels Based on Dicer, Drosha, or Both

We have also discovered that the expression levels of Dicer and Drosha in a neuroblastoma patient are closely related to that patient's risk level. Thus, Dicer, Drosha, and their combination serve as indicators for assessing a neuroblastoma patient's risk level.

To perform this assessment method, the expression levels of Dicer and Drosha can be determined following the above-described method and normalized against the expression level of an internal control (e.g., GAPDH or β-actin). The data thus obtained is processed by a computational program to produce a signature characterizing the expression level of Dicer, Drosha, or the combination of Dicer and Drosha. This signature is compared with a cut-off value that distinguishes high-risk neuroblastoma patients from low-risk neuroblastoma patients. In one example, this cut-off value is obtained by analyzing the expression levels of Dicer and Drosha of high-risk and low-risk patients via student t-test. If the signature is greater than the cut-off value, representing high expression of Dicer or Drosha, the patient is determined as having a low risk level; if it is lower than the cut-off value, representing low expression of Dicer or Drosha, the patient is determined as having a high risk.

When the just-described method uses Dicer as the indicator, it can be applied to neuroblastoma patients with no MYCN amplification.

Inhibiting Neuroblastoma Cell Growth with Dicer or Drosha

Also within the scope of this invention is a method of inhibiting neuroblastoma cell growth with Dicer or Drosha. In one example, a polypeptide including the amino acid sequence of Dicer or Drosha is used in this method. In another example, a nucleic acid encoding the just-mentioned polypeptide is used. Dicer or Drosha can be naturally-occurring proteins from human, swine, mouse, rat, or other species. It also can be a functional variant of any of the naturally-occurring proteins, i.e., having a sequence at least 85% (e.g., 90%, 95%, or 98%) to its wild-type counterpart.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Any of the above-mentioned polypeptides or nucleic acids can be prepared via conventional methods, e.g., recombinant technology. It can then mixed with a pharmaceutically acceptable carrier to form a pharmaceutical composition. A “pharmaceutically acceptable carrier” is a carrier compatible with the activity of the fusion protein (and preferably, stabilizing the activity of the fusion protein) and not deleterious to the subject to be treated. Examples of carriers include but are not limited to water, saline, dextrose, glycerol, ethanol, and combinations thereof. The pharmaceutical composition may further contain minor amounts of auxiliary substances such as wetting or emulsifying agents, pH buffering agents.

An effective amount of the pharmaceutical composition can be administered to a neuroblastoma patient via a conventional route to suppress neuroblastoma cell growth. When a Dicer or Drosha polypeptide is used, it can be dissolved or suspended in the carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the subject's illness; the subject's size, weight, surface area, age, and sex; other drugs being administered; and the judgment of the attending physician. Suitable dosages are in the range of 0.01-100.0 mg/kg. Wide variations in the needed dosage are to be expected in view of the variety of compositions available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the composition in a suitable delivery vehicle (e.g., polymeric microparticles or implantable devices) may increase the efficiency of delivery, particularly for oral delivery.

The just-described pharmaceutical composition can be formulated into dosage forms for different administration routes utilizing conventional methods. For example, it can be formulated in a capsule, a gel seal, or a tablet for oral administration. Capsules can contain any standard pharmaceutically acceptable materials such as gelatin or cellulose. Tablets can be formulated in accordance with conventional procedures by compressing mixtures of the composition with a solid carrier and a lubricant. Examples of solid carriers include starch and sugar bentonite. The composition can also be administered in a form of a hard shell tablet or a capsule containing a binder, e.g., lactose or mannitol, a conventional filler, and a tableting agent. The pharmaceutical composition can be administered via the parenteral route. Examples of parenteral dosage forms include aqueous solutions, isotonic saline or 5% glucose of the active agent, or other well-known pharmaceutically acceptable excipient. Cyclodextrins, or other solubilizing agents well known to those familiar with the art, can be utilized as pharmaceutical excipients for delivery of the therapeutic agent.

The efficacy of the pharmaceutical composition described herein can be evaluated both in vitro and in vivo. Based on the results, an appropriate dosage range and administration route can be determined.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### Example 1

### Identification of miRNAs Differentially Expressed in Different Staged Neuroblastoma Tumor

To determine miRNA expression profiles for neuroblastoma tumors in various International Neuroblastoma Staging System (INSS) stages, total mRNAs were collected from primary neuroblastoma tumors of 66 patients (Caucasians). The clinicopathological information of these 66 patients is summarized in Table 3 below:

TABLE 3
Clinicophthological Information of Neuroblastoma Patients

| variable | Cases (N = 66) |
|---|---|
| INSS stage | |
| Stage 1 | 7 |
| Stage 2 | 18 |
| Stage 3 | 12 |
| Stage 4 | 22 |
| Stage 4S | 7 |
| MYCN | |
| Amplification | 13 |
| Non Amplification | 53 |
| Risk | |
| Low | 31 |
| Intermediate | 10 |
| High | 25 |
| Survival | |
| Alive | 49 |
| Dead | 17 |
| Event | |
| no event | 45 |
| event | 21 |
| Age at diagnosis | |
| <1.5 year | 43 |
| ≧1.5 year, <5 year | 18 |
| ≧5 year | 5 |
| Shimada histology | |
| Favorable | 29 |
| Unfavorable | 18 |
| Missing | 19 |
| Sex | |
| Male | 23 |
| Female | 36 |
| missing | 7 |
| Sample source | |
| COG (Children’s Oncology Group) | 35 |
| POG (Pediatric Oncology Group) | 21 |
| CHTN (Cooperative Human Tissue Network) | 10 |

The expression levels of 162 miRNAs in the 66 primary neuroblastoma tumors were quantified by real-time PCR using the TaqMan MicroRNA Assays Human Panel-Early Access Kit (Applied Biosystems, Foster City, Calif.), according to the manufacturer's protocol. Briefly, to amplified each miRNA, 2.5 ng of total RNA (in 15 μl volume) was subjected to gene-specific reverse transcription, using the TaqMan microRNA Reverse Transcription Kit, followed by q-PCR amplification using miRNA-specific primers, using the 7300 Sequence Detection System (Applied Biosystems). Data indicating threshold cycle (Ct) values of the miRNAs was determined by default threshold setting (0.2) and was normalized against the Ct value of U6 rRNA, a common internal control for miRNA quantification assays. See Chen et al., Cancer Research 67(3):976-983, 2007; and Jiang et al., Nucl. Acids. Res. 33(17):5394-5403, 2005. The Ct values higher or equal to 36, were adjusted to 36, a value representing no expression. This data analysis was performed using GENESPRING software (version 7.2, Silicon Genetics, Redwood City, Calif.).

To identify miRNAs of interest, whose expression levels correspond to clinicopathological factors, median normalization on each miRNA was first performed followed by subsequent statistical comparisons using ANOVA with the Benjamin and Hochberg correction for false positive reduction. Hierarchical clustering for the miRNAs or the clinicopathological factors was generated by standard correlation.

Further analyses to identify miRNAs that are differentially expressed in different risk groups were performed by using algorithm of Prediction Analysis of Microarray (PAM), following the method described in Tibshirani et al., PNAS 99(10):6567-6572, 2002.

miRNA expression profiles were generated using unsupervised agglomerative hierarchical clustering. Global down-regulation of miRNA expression (139 out of the 162 miRNAs) was observed in advanced neuroblastoma tumors (i.e., INSS stage 4), particularly in advanced tumors with MYCN amplification, as relative to miRNA expression in tumors in the other INSS stages.

Applying the method of the nearest shrunken centroids as implemented in PAM (see Tibshirani), 33 miRNAs were identified to be differentially expressed in tumors in different INSS stages. The prediction error was calculated by means of 10-fold cross-validation and the 33 miRNAs listed in Table 1, which yielded the minimum misclassification error (i.e., threshold=2.0) were identified as differentially expressed in different tumor stages. Table 4 below lists 27 of the 33 miRNAs, whose expression levels were found to be associated with neuroblastoma risk levels.

TABLE 4
miRNAs That Are Differentially Expressed In High-Risk and Low-Risk
Neuroblastoma Patients

| | | | | | PAM Score^{b} | |
|---|---|---|---|---|---|---|
| | | | Fold | | High-Risk | Low-Risk |
| No. | miRNA | Location | Change* | P value^{a} | (n = 10) | (n = 31) |
| 1 | hsa-miR-149 | 2q37.3 | −3.8 | 1.66E−05 | −0.0918 | 0 |
| 2 | hsa-miR-129 | 7q32.1/ | −8.5 | 3.44E−05 | −0.0892 | 0.0194 |
| | | 11p11.2 | | | | |
| 3 | hsa-miR-27b | 9q22.32 | −5.7 | 1.66E−05 | −0.0835 | 0.0074 |
| 4 | hsa-miR-23b | 9q22.1 | −4.4 | 1.66E−05 | −0.0828 | 0 |
| 5 | hsa-miR-190 | 15q22.2 | −3.8 | 1.05E−04 | −0.0822 | 0 |
| 6 | hsa-miR-128a | 2q21 | −4.8 | 1.66E−05 | −0.0746 | 0 |
| 7 | hsa-miR-15a | 13q14 | −6.5 | 3.56E−05 | −0.0661 | 0 |
| 8 | hsa-miR-148a | 7p15.2 | −5.3 | 1.53E−04 | −0.0656 | 0.0364 |
| 9 | hsa-miR-137 | 1p21.3 | −5.1 | 1.16E−04 | −0.0616 | 0 |
| 10 | hsa-miR-30c | 1p34.2/ | −4.3 | 1.66E−05 | −0.0514 | 0 |
| | | 6q13 | | | | |
| 11 | hsa-miR-197 | 1p13 | −4.1 | 2.13E−05 | −0.0418 | 0 |
| 12 | hsa-miR-195 | 17p13 | −4.4 | 2.29E−04 | −0.0297 | 0 |
| 13 | hsa-miR-26b | 2q35 | −4.6 | 1.66E−05 | −0.0273 | 0 |
| 14 | hsa-miR-21 | 17q23.2 | −3.7 | 5.52E−05 | −0.0267 | 0 |
| 15 | hsa-miR-30b | 8q24.22 | −3.4 | 1.66E−05 | −0.0264 | 0 |
| 16 | hsa-miR-135a | 3p21.1/ | −4.0 | 2.40E−04 | −0.0259 | 0 |
| | | 12q23.1 | | | | |
| 17 | hsa-miR-126 | 9q34.3 | −4.2 | 6.44E−05 | −0.0215 | 0.014 |
| 18 | hsa-miR-95 | 4p16 | −3.7 | 8.63E−05 | −0.0206 | 0 |
| 19 | hsa-miR-142-5p | 17q23 | −3.8 | 8.26E−04 | 0 | 0.0201 |
| 20 | hsa-miR-128b | 3p22 | −4.0 | 1.39E−04 | −0.0171 | 0 |
| 21 | hsa-miR-98 | xp11.2 | −4.7 | 8.69E−05 | −0.0137 | 0 |
| 22 | hsa-miR-142-3p | 17q23 | −4.4 | 8.26E−04 | 0 | 0.0131 |
| 23 | hsa-miR-340 | 5q35.3 | −2.5 | 3.44E−05 | −0.0079 | 0 |
| 24 | hsa-miR-30e | 1p34.2 | −4.6 | 1.94E−05 | −0.0071 | 0 |
| 25 | hsa-miR-331 | 12q22 | −3.0 | 3.44E−05 | −0.004 | 0 |
| 26 | hsa-miR-140 | 16q22.1 | −2.9 | 3.56E−05 | −0.0032 | 0 |
| 27 | hsa-miR-324-5p | 17p13.1 | −3.6 | 1.87E−04 | −0.0022 | 0 |

*miRNA expression level fold changes in high-risk patients as compared to low-risk patients
^{a}obtained by ANOVA with Welch t test provided in the GeneSpring software package
^{b}Centroid scores obtained by PAM analysis

As shown in Table 4 above, the expression level of each of the listed miRNA was much lower in high-risk patients than in low-risk patients (i.e., 2.5 to 8.5 fold lower).

### Example 2

### Distinguishing High-Risk Neuroblastoma Patients from Low-Risk Patients Based on a 27-miRNA Signature

Via PAM analysis, the expression levels of the 27 miRNAs listed in Table 1 above were found to be associated with a neuroblastoma patient's risk level. More specifically, based on this 27-miRNA signature of the patient as determined by PAM analysis, 23 out of 25 high-risk patients and 26 out of 31 low-risk patients were correctly classified into the proper risk group, with accuracy of 92% and 84%, respectively. Based on the same miRNA signature, 9 of 10 intermediate-risk samples were classified as low-risk patients. These patients indeed exhibited good clinical outcomes.

The expression levels of the 27 miRNAs in the 66 neuroblastoma patients mentioned in Example 1 above were also subjected to PAM analysis. Based on the 27-miRNA signature of these patients, 34 patients were determined as low-risk patient, 5 as intermediate-risk patients, and 22 as high-risk patients. None of the 34 low-risk patients had MYCN amplification in their neuroblastoma and 28 out of the 34 patient were diagnosed at <1.5 yr. All of these patients survived. Upon clinical staging, these 34 patients were classified as in INSS stage 1, 2, 3, or 4S. Except for stage 3 patients, those in the other stages are classified as low-risk patients based on the current Children's Oncology Group (COG) system. See Table 2 above.

Most of the patients who were determined as intermediate-risk or high-risk patients in this study bore advanced tumors. Among the five intermediate-risk patients, 3 were found to have MYCN amplification disease and with a poorer prognosis. All stage 4 patients (high risk based on COG) were assigned to the high-risk group. Of the 9 patients having MYCN amplification, 7 were determined as high-risk patients.

The above results indicate that the miRNA signature constituting the 27 miRNAs listed in Table 4 above is a reliable marker for determining the risk level of a neuroblastoma patient.

### Example 3

### Determining Risk Levels of Neuroblastoma Patients Based on the Expression Levels of Dicer or Drosha

Real-time RT-PCR was performed to determine the levels of Dicer and Drosha in 65 of the 66 neuroblastoma tumor samples mentioned in Example 1 above, following the procedures described in Karube et al. Cancer Sci. 96(2):111-115, 2005). Briefly, 10 ng total RNAs isolated from each of the neuroblastoma samples of the 66 patients were reverse transcribed to cDNAs using SuperScript™ First-Strand Synthesis System with random hexamer primers (Invitrogen). The cDNAs were then subjected to real-time quantitative PCR in 1X SYBR Green Master Mix (Applied Biosystems), using and Dicer-, Drosha- or GAPDH-specific primers as described in Karube et al., Cabcer Sci. 96(2):111-115, 2005 and Applied Biosystems PRISM 7300-HT. All reactions were performed in triplicate. The expression levels of Dicer and Drosha thus obtained were normalized against the expression level of GAPDH in the same sample.

The expression levels of Dicer and Drosha were then subjected to student t-test to determine a cut-off value that has the highest potential for discriminating two distinct groups, i.e., high-risk group and low-risk group. The results show that the cut-off value for Dicer is about −4.5 and that of Drosha is about −5.13.

Low expression of Drosha was observed in 82% of neuroblastoma patients in stage 4, in 84% high-risk patients, and in 85% patients bearing MYCN amplification (a high risk indicator). Similarly, the expression level of Dicer in stage 4 tumors were significantly lower that that in tumors in other stages, particularly in stage 4S (p<0.001). Low expression of Dicer was found to be associated with other high-risk indicators, such as unfavaorable age at diagnosis, later disease stage, MYCN amplification, and Shimada histology (p<0.001, p<0.038, p<0.013, and p<0.004, respectively).

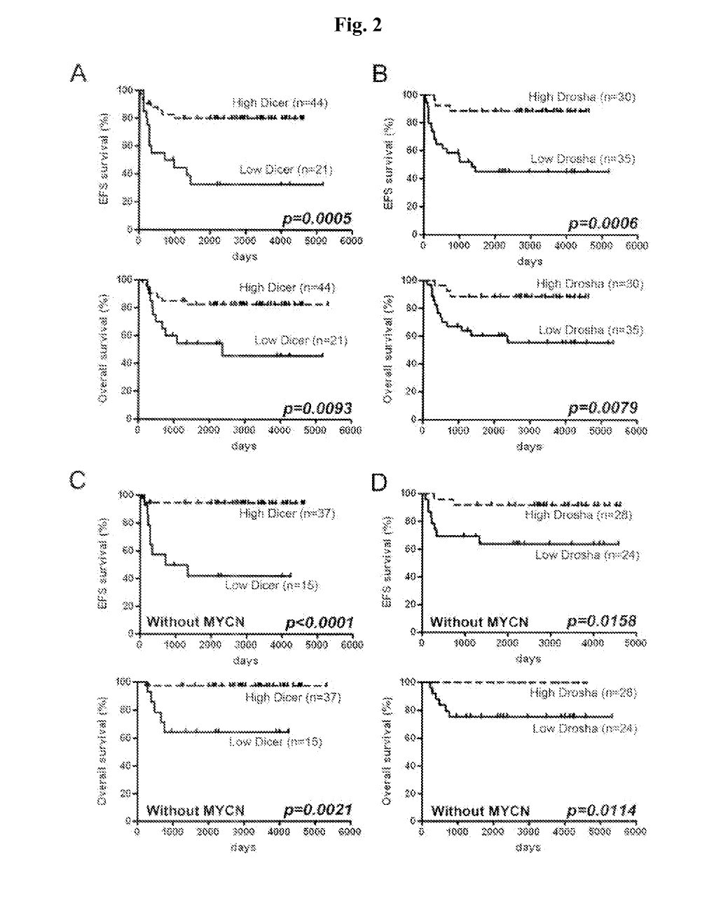

The expression level of Dicer or Drosha was also found to be associated with a patient's survival rate. More specifically, the results obtained from Kaplan-Meier survival analyses show that neuroblastoma patients with low expression of Dicer had a significantly lower event-free survival rate than those with high expression of Dicer (32.4% vs. 79.9%, p=0.0005); and the overall survival rate of neuroblastoma patients with low Dicer expression was significantly lower than those with high Dicer expression (45.5 vs. 82.2%, p=0.0093). See FIG. 2, panel (a). Event-free means that no tumor recurrence or complications resulting from treatment. Likewise, the expression level of Drosha was also found to be associated with a patient's survival rate. Namely, the level of Drosha expression was much lower in patients with a low event-free survival rate than in patients with a high event-free rate (44.7% vs. 88.5%, p=0.0006). The overall survival rate of patients with low Drosha expression was significantly lower that of patients with high Drosha expression (55.4% vs. 88.5%, p=0.0079). See FIG. 2, panel (b).

Further, Univariate Cox regression analysis of various risk factors, i.e., clinical stage, expression levels of Dicer and Drosha, and MYCN amplification status, revealed that low expression of Dicer or Drosha, independently, was predictive of lower event-free and overall survival rates of neuroblastoma patients. The correlations between Dicer and Drosha expression levels and various clinicopathologic characteristics are summarized in Table 5 below:

TABLE 5
Correlations Between Levels of Dicer/Drosha and Clinicopathologic
Characteristics

| | | Dicer | | | Drosha | | |
|---|---|---|---|---|---|---|---|
| Characteristics | cases | High | Low | p^{a} | High | Low | p^{a} |
| Sex | | | | | | | |
| Male | 35 | 23 | 12 | 1.0 | 17 | 18 | 1.0 |
| Female | 23 | 15 | 8 | | 12 | 1 | |
| Age at diagnosis | | | | | | | |
| ≦1 year | 32 | 29 | 3 | <0.001 | 19 | 13 | 0.048 |
| >1 year | 33 | 15 | 18 | | 11 | 22 | |
| INSS Stage | | | | | | | |
| 1 | 7 | 4 | 3 | 0.038^{b} | 5 | 2 | 0.006^{b} |
| 2 | 17 | 14 | 3 | | 10 | 7 | |
| 3 | 12 | 9 | 3 | | 6 | 6 | |
| 4 | 22 | 10 | 12 | | 4 | 18 | |
| 4S | 7 | 7 | 0 | | 5 | 2 | |
| Risk | | | | | | | |
| Low and | 40 | 32 | 8 | 0.013 | 26 | 14 | <0.001 |
| intermediate | | | | | | | |
| High | 25 | 12 | 13 | | 4 | 21 | |
| Histology | | | | | | | |
| favorable | 29 | 24 | 5 | 0.004 | 16 | 13 | 0.079 |
| unfavorable | 18 | 7 | 11 | | 5 | 13 | |
| MYCN | | | | | | | |
| Non-amplified | 52 | 37 | 15 | 0.321 | 28 | 24 | 0.015 |
| Amplified | 13 | 7 | 6 | | 2 | 11 | |

^{a}Two-sided Fisher's exact test
^{b}compare stage 1, 2 and 4S with stage 3 and 4

Finally, the expression levels of Dicer in 52 patients bearing neuroblastoma tumors with no MYCN amplification was subjected to univariate and multivariate Cox regression model analysis. Low Dicer expression was found to be associated with poor clinical outcome while high Dicer expression was associated with high event-free and over-all survival rates. See FIG. 2. Based on the result, Dicer was identified as an independent indicator for predicting the overall survival rates/risk levels of neuroblastoma patients with no MYCN amplification.

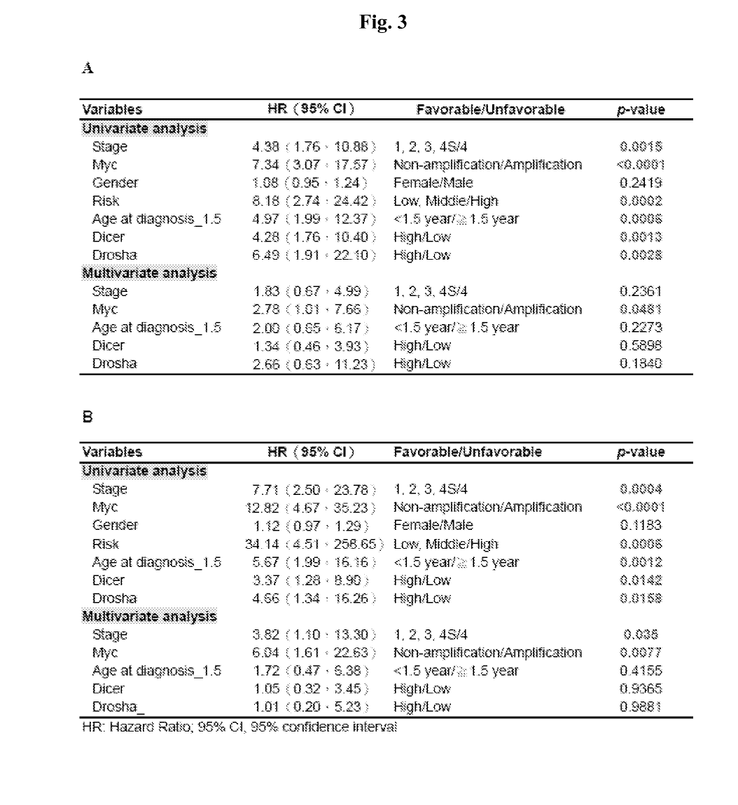

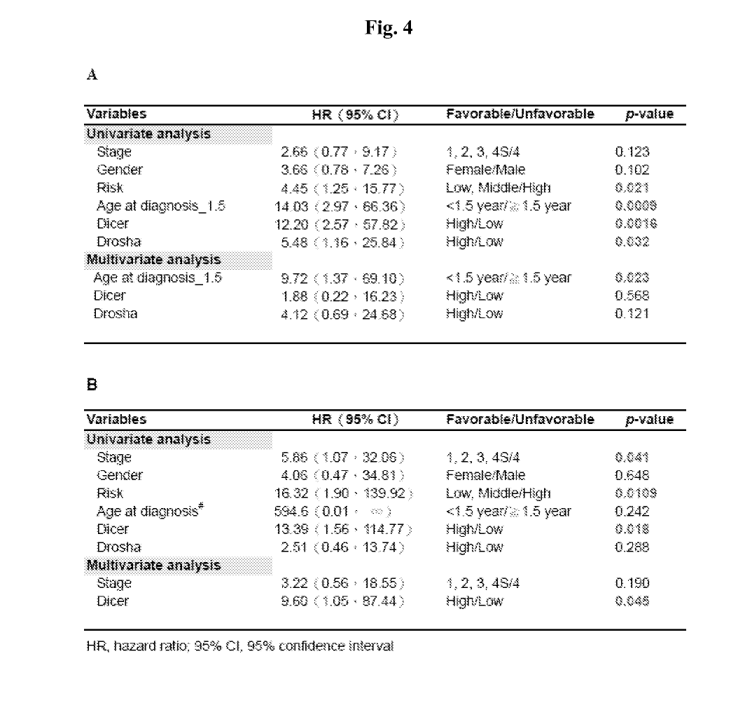

The correlations between Dicer/Drosha, as well as other clinical factors, and the patients' clinical outcomes are shown in FIGS. 3 and 4.

### Example 4

### Assessing Risk Levels of Neuroblastoma Patients Based on a Signature of 15 Biomarkers

Using PNNSolution, the Multivariate Data Clustering and Classification System provided by PNN Technologies, Inc., a unique signature consisting of 15 biomarkers, i.e., miRNAs hsa-miRNAs-29a, hsa-miRNAs-30c, hsa-miRNAs-30e, hsa-miRNAs-95, hsa-miRNAs-128a, hsa-miRNAs-128b, hsa-miRNAs-135a, hsa-miRNAs-135b, hsa-miRNAs-137, hsa-miRNAs-138, hsa-miRNAs-148a, and hsa-miRNAs-195, Dicer, Drosha, and age at diagnosis, was identified as an indicator for classifying 65 neuroblastoma patients into four risk groups, each having a risk pattern of Patterns A-D. See FIG. 1. The patients having a risk pattern of Pattern A are all high-risk patients with a death rate of 74%. The patients having a risk pattern of Pattern B can be low, intermediate, or high-risk patients with the death rate of 12%. Pattern C and Pattern D patients were in either low or intermediate risk levels with 0% death rate.

Relying on this 15-biomarker signature, the survival/death probability of neuroblastoma patients was successfully predicted using the PNNSolution system. The accuracy of this study is 83%.

### Example 5

### Down-Regulation of Dicer or Drosha Promoted Neuroblastoma Cell Proliferation

Neuroblastoma cell lines, Be2C. NMB7, and NB5, were cultured under the conditions described in Diccianni et al., International Journal of Cancer, 80(1):145-154, 1999 and Lin et al., Oncogene 26(49):7017-7027, 2007. These cells were transfected with plasmids TRCN0000051262, TRCN0000022253, and TRCN0000072243 using Lipofectamine 2000 (Invitrogen, Carlsbad, Calif.) according to the manufacturer's instructions. These plasmids, obtained from the National RNAi Core Facility, Genomic Research Center, Academia Sinica, Taiwa, were designed for expressing shRNAs targeting human Dicer, human Drosha, and firefly luciferase (as the negative control). The expression levels of Dicer and Drosha, as well as certain miRNAs, in the transfected cells were determined by routine methods.

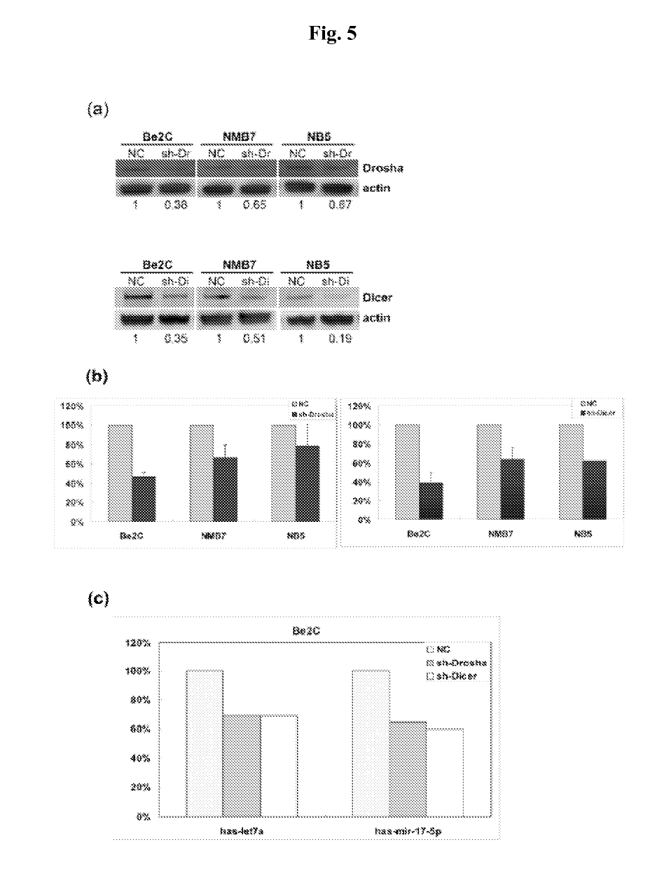

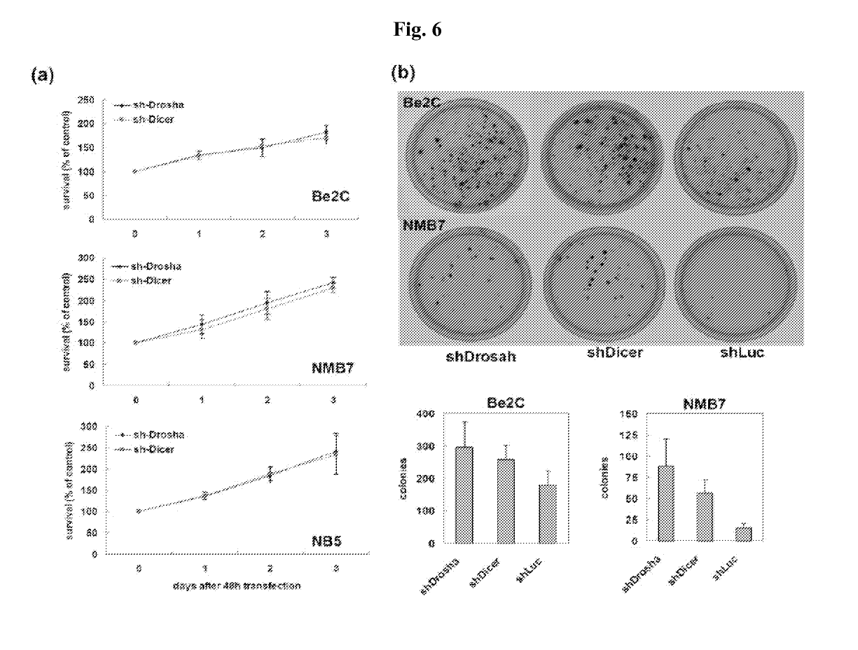

As shown in FIG. 5, panels A, B, and C, expression of shRNAs targeting Dicer and Drosha successfully reduced Dicer/Drosha expression in transfected neuroblastoma cells. Low expression of Dicer/Drosha also resulted in reduced expression of miRNAs hsa-let7a and hsa-mir-17-5p. In comparison to the negative control cells, cells expressing shRNAs targeting Dicer or Drosha proliferated more rapidly when cultured in a liquid medium and produced more and larger colonies when cultured on a solid medium. See FIG. 6. These findings indicate that down-regulation of Dicer or Drosha promotes neuroblastoma cell proliferation. It is therefore suggested that up-regulation of Dicer or Drosha would inhibit neuroblastoma cell growth.

### Example 6

### Survival-Death Probability Assessment of Neuroblastoma Patients Based on a 20-miRNA Signature

Using a Probabililic Neural Network (PNN) model provided by PNN Technologies, Inc., a miRNA signature constituting the 20 miRNAs listed in Table 6 below was identified as a reliable marker for predicting a neuroblastoma patient's survival/death probability.

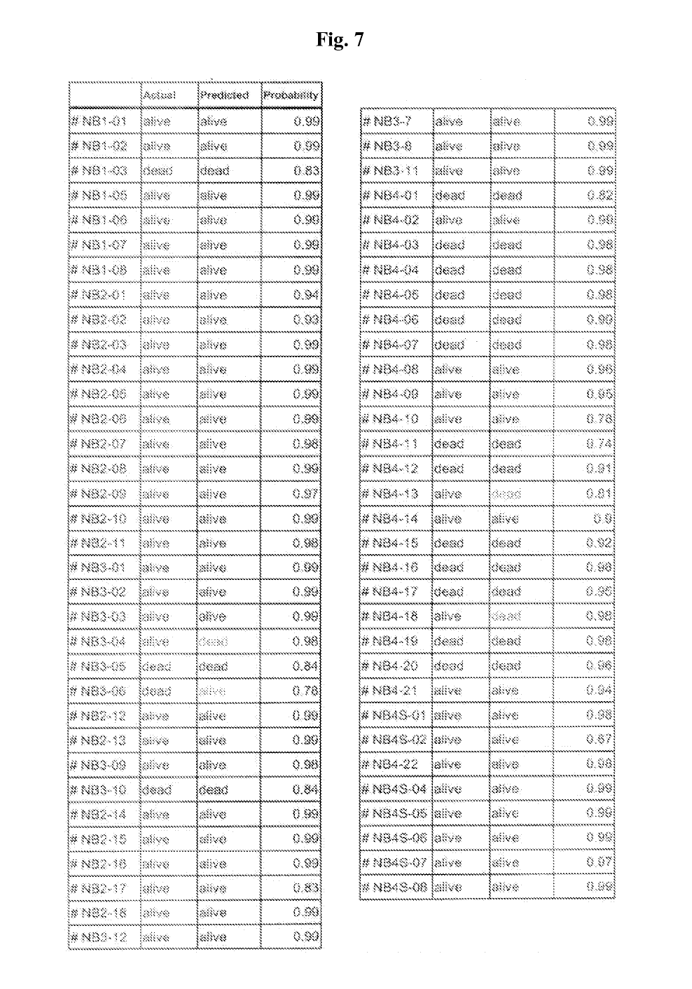

Based on this 20-miRNA signature, 62 out of the 66 neuroblastoma patients mentioned in Example 1 above were correctly determined for their survival/death status. See FIG. 7. The prediction accuracy is about 94%.

TABLE 6
MiRNAs for Predicting Survival/Death
Probability of Neuroblastoma Patients

| | | | fold change | |
|---|---|---|---|---|
| | miR | location | (dead/alive) | PNN score |
| 1 | hsa-miR-26a | 3p21 | −2.82 | 0.0819025 |
| 2 | hsa-miR-26b | 2q35 | −3.84 | 0.0710598 |
| 3 | hsa-miR-27b | 9q22.32 | −3.88 | 0.0949939 |
| 4 | hsa-miR-30a-3p | 6q12-13 | −3.44 | 0.0571802 |
| 5 | hsa-miR-30e | 1p34.2 | −4.14 | 0.0805412 |
| 6 | hsa-miR-95 | 4p16 | −3.64 | 0.0863976 |
| 7 | hsa-miR-128a | 2q21 | −5.45 | 0.140485 |
| 8 | hsa-miR-128b | 3p22 | −4.42 | 0.132102 |
| 9 | hsa-miR-129 | 7q32.1/11p11.2 | −10.53 | 0.138483 |
| 10 | hsa-miR-137 | 1p21.3 | −6.50 | 0.105999 |
| 11 | hsa-miR-146 | 5q34 | −5.13 | 0.102684 |
| 12 | hsa-miR-148a | 7p15.2 | −4.71 | 0.089935 |
| 13 | hsa-miR-149 | 2q37.3 | −5.48 | 0.141809 |
| 14 | hsa-miR-152 | 17q21 | −3.17 | 0.0600925 |
| 15 | hsa-miR-186 | 1p31 | −2.92 | 0.0636353 |
| 16 | hsa-miR-190 | 15q22.2 | −4.31 | 0.123051 |
| 17 | hsa-miR-197 | 1p13 | −4.32 | 0.0999558 |
| 18 | hsa-miR-324-5p | 17p13.1 | −4.04 | 0.113054 |
| 19 | hsa-miR-331 | 12q22 | −3.79 | 0.0902461 |
| 20 | hsa-miR-335 | 7q32.2 | −4.20 | 0.0841078 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A method of determining the risk level of a neuroblastoma patient, comprising
obtaining a set of data indicating (i) the expression levels of microRNAs hsa-miRNAs-29a, hsa-miRNAs-30c, hsa-miRNAs-30e, hsa-miRNAs-95, hsa-miRNAs-128a, hsa-miRNAs-128b, hsa-miRNAs-135a, hsa-miRNAs-135b, hsa-miRNAs-137, hsa-miRNAs-138, hsa-miRNAs-148a, and hsa-miRNAs-195 in a neuroblastoma sample of the patient, (ii) the expression levels of Dicer and Drosha in the sample, and (iii) the patient's age at diagnosis,
processing the set of data by computational analysis to determine a risk pattern, and
assessing the patient's risk level based on the risk pattern,
wherein the risk pattern being Pattern A indicates that the patient has a high risk level, the risk pattern being Pattern C indicates that the patient has a low risk level, and the risk pattern being Pattern D indicates that the patient has a medium or low risk level.

2. The method of claim 1, wherein the neuroblastoma patient is free of clinical staging and any other risk assessment.

3. The method of claim 1, wherein the expression levels of the microRNAs, Dicer, and Drosha are determined by real-time PCR.

4. A method of assessing the risk level of a neuroblastoma patient, comprising:
obtaining a set of data indicating expression levels of microRNAs hsa-miR-149, hsa-miR-129, hsa-miR-27b, hsa-miR-23b, hsa-miR-190, hsa-miR-128a, hsa-miR-15a, hsa-miR-148a, hsa-miR-137, hsa-miR-30c, hsa-miR-197, hsa-miR-195, hsa-miR-26b, hsa-miR-21, hsa-miR-30b, hsa-miR-135a, hsa-miR-126, hsa-miR-95, hsa-miR-142-5p, hsa-miR-128b, hsa-miR-98, hsa-miR-142-3p, hsa-miR-340, hsa-miR-30e, hsa-miR-331, hsa-miR-140, and hsa-miR-324-5p in a neuroblastoma sample of a patient,
processing the set of data by computational analysis to determine a microRNA signature that characterizes the expression profile of the microRNAs, and
assessing the risk level of the patient based on the microRNA signature, wherein a signature representing low expression of the microRNAs indicates that the patient is a high-risk neuroblastoma patient and a signature representing high expression of the microRNAs indicates that the patient is a low-risk neuroblastoma patient.

5. The method of claim 4, wherein the obtaining step is performed by determining the expression levels of the microRNAs via real-time PCR.

6. A method of assessing the risk level of a neuroblastoma patient, comprising:
obtaining a set of data indicating the expression level of one or more microRNAs in a neuroblastoma sample of a patient, the one or more microRNAs being selected from the group consisting of hsa-miR-23b, hsa-miR-128a, hsa-miR-15a, hsa-miR-148a, hsa-miR-197, hsa-miR-195, hsa-miR-26b, hsa-miR-21, hsa-miR-135a, hsa-miR-126, hsa-miR-95, hsa-miR-142-5p, hsa-miR-128b, hsa-miR-98, hsa-miR-142-3p, hsa-miR-340, and hsa-miR-140,
processing the set of data by computational analysis to determine a microRNA signature that characterizes the expression profile of the one or more microRNAs, and
assessing the risk level of the patient based on the microRNA signature, wherein a signature representing low expression of the microRNAs indicates that the patient is a high-risk neuroblastoma patient and a signature representing high expression of the microRNAs indicates that the patient is a low-risk neuroblastoma patient.

7. The method of claim 6, wherein the obtaining step is performed by determining the expression level(s) of the microRNA(s) via real-time PCR.

8. A method of determining the risk level of a neuroblastoma patient, comprising:
obtaining a first set of data indicating the expression level of one or more microRNAs in a neuroblastoma sample of a patient, the one or more microRNAs being selected from a first microRNA group including hsa-miR-23b, hsa-miR-128a, hsa-miR-15a, hsa-miR-148a, hsa-miR-197, hsa-miR-195, hsa-miR-26b, hsa-miR-21, hsa-miR-135a, hsa-miR-126, hsa-miR-95, hsa-miR-142-5p, hsa-miR-128b, hsa-miR-98, hsa-miR-142-3p, hsa-miR-340, and hsa-miR-140,
obtaining a second set of data indicating the expression level of one or more microRNAs in the neuroblastoma sample, the one or more microRNAs being selected from a second microRNA group including hsa-miR-149, hsa-miR-129, hsa-miR-27b, hsa-miR-190, hsa-miR-137, hsa-miR-30c, hsa-miR-30b, hsa-miR-30e, hsa-miR-331, and hsa-miR-324-5p,
processing the first and second sets of data by computational analysis to determine a microRNA signature that characterizes the expression profile of the one or more microRNAs in both the first and second microRNA groups, and
assessing the risk level of the patient based on the microRNA signature, wherein a signature representing low expression of the microRNAs indicates that the patient is a high-risk neuroblastoma patient and a signature representing high expression of the microRNAs indicates that the patient is a low-risk neuroblastoma patient.

9. The method of claim 8, wherein the obtaining steps are performed by determining the expression levels of the one or more microRNAs in both the first and second microRNA groups by real-time PCR.

10. A method of assessing the risk level of a neuroblastoma patient, comprising
obtaining a set of data indicating the expression level(s) of Dicer, Drosha, or both in a neuroblastoma sample of the patient,
processing the set of data by computational analysis to determining a signature that characterizes the expression profile of Dicer, Drosha, or both, and
assessing the risk level of the patient based on the signature, wherein a signature representing low expression of Dicer, Drosha, or both indicates that the patient is a high-risk neuroblastoma patient and a signature representing high expression of Dicer, Drosha, or both indicates that the patient is a low-risk neuroblastoma patient.

11. The method of claim 10, wherein the obtaining step is performed by determining the expression level(s) of Dicer, Drosha, or both via real-time PCR.

12. The method of claim 10, wherein the obtaining step is performed by determining the expression level of Dicer.

13. The method of claim 12, wherein the patient bears a neuroblastoma tumor, in which MYCN is not amplified.

14. The method of claim 13, wherein the expression level of Dicer is determined by real-time PCR.

15. A method of inhibiting neuroblastoma cell growth, comprising administering to a patient in need thereof an effective amount of a composition containing (i) a polypeptide including the amino acid sequence of Dicer or Drosha, or a nucleotide sequence encoding the polypeptide, and (ii) a pharmaceutically acceptable carrier.

16. The method of claim 15, wherein the composition contains Dicer.

17. The method of claim 15, wherein the composition contains Drosha.

18. A method of assessing the survival/death probability of a neuroblastoma patient, comprising:
obtaining a set of data indicating expression levels of microRNAs hsa-miR-26a, hsa-miR-26b, hsa-miR-27b, hsa-miR-30a-3p, hsa-miR-30e, hsa-miR-95, hsa-miR-128a, hsa-miR-128b, hsa-miR-129, hsa-miR-137, hsa-miR-146, hsa-miR-148a, hsa-miR-149, hsa-miR-152, hsa-miR-186, hsa-miR-190, hsa-miR-197, hsa-miR-324-5p, hsa-miR-331, and hsa-miR-335 in a neuroblastoma sample of a patient,
processing the set of data by computational analysis to determine a microRNA signature that characterizes the expression profile of the microRNAs, and
assessing the survival/death probability of the patient based on the microRNA signature, wherein a signature representing low expression of the microRNAs indicates that the patient has a high probability of survival and a signature representing high expression of the microRNAs indicates that the patient has a low probability of survival.

19. The method of claim 18, wherein the obtaining step is performed by determining the expression levels of the 20 microRNAs by real-time PCR.
